(19) United States
(12) Patent Application Publication
(10) Pub. No.: US 2013/0204277 A1
(43) Pub. Date: Aug. 8, 2013

(54) THREE-DIMENSIONAL SURGICAL IMPLANT

(71) Applicants: Roland Fabry, Metz (FR); Greg Tebbe, Ridgefield, CT (US)

(72) Inventors: Roland Fabry, Metz (FR); Greg Tebbe, Ridgefield, CT (US)

(73) Assignee: TYCO HEALTHCARE GROUP LP, New Haven, CT (US)

(21) Appl. No.: 13/636,886

(22) PCT Filed: Mar. 24, 2011
(86) PCT No.: PCT/US11/29826

§ 371 Date: Jan. 25, 2013

Related U.S. Application Data
(60) Provisional application No. 61/317,009, filed on Mar. 24, 2010.

(51) Int. Cl. A61F 2/00

(52) U.S. Cl. CPC A61F 2/0063
USPC 606/151; 28/143

(57) ABSTRACT

Three-dimensional surgical implants include a grip-type knit mesh folded into a three-dimensional structure. Spiked naps provided on the mesh grip pores on the mesh to hold the implant in the three-dimensional structure.

### BACKGROUND

1. Technical Field

The present disclosure relates to implants or surgical meshes and, more particularly, to meshes that have a grip-type knit mesh knit and a three-dimensional structure.

2. Description of the Related Art

Surgical meshes formed from degradable or non-degradable materials for use during both open and minimally invasive surgeries are known. These meshes are typically flat fibrous material that a surgeon places over a defect, such as a tear in tissue, as reinforcement. The surgeon then secures the mesh in place with a surgical fastener, such as a staple, clip, tack, suture or the like.

Meshes exhibiting structures other than a planar or flat structure are also known. These meshes form a plug to fill the defect. In some cases, these meshes are preformed from permanent rigid materials with pleats to create some form of flexibility. These permanent meshes can also require a separate flat mesh overlay to reinforce the defect.

Surgical meshes formed from non-degradable materials can be rigid. Rigid surgical meshes have benefits in hernia repair, for example, a rigid hernia mesh keeps the hernia sac retracted, is quicker and easier to use, and is inserted using an easily reproducible procedure. However, the non-degradable materials result in permanent foreign material inside a patient's body. The heavy non-degradable materials used to form rigid meshes also have small pore sizes, which can inhibit tissue in-growth.

Surgical meshes formed from degradable materials may produce a soft, pliant surgical mesh. The level of flexibility of a pliant mesh is controlled by the materials used to form the mesh and the weave or knitting of the mesh. For example, a large pore mesh formed from lightweight degradable materials has enhanced tissue in-growth and reduced inflammatory response following implantation; it also results in less scarring than a heavyweight, small pore mesh. A soft, pliant mesh will form to the abdominal wall of the patient's body and flex more naturally with the movement of the abdominal wall following implantation. Due to the more natural action of a flexible, pliant mesh the patient typically experiences less postoperative pain and improved comfort. However, meshes made solely from degradable material may not be suitable for long term hernia repair.

It would be advantageous to provide a surgical mesh formed of both non-degradable and degradable materials so as to produce a soft, pliant mesh providing improved comfort and less postoperative pain for the patient. It would also be advantageous to provide a surgical mesh that can be formed or reformed into a three-dimensional structure needed to fit the defect.

In particular, it would be advantageous to provide a surgical mesh that forms and maintains a three-dimensional structure, exhibits the flexibility of a degradable mesh and the strength of a non-degradable mesh, leaves little permanent foreign material inside a patient's body, and secures itself within the defect.

### SUMMARY

The present disclosure is directed to a three-dimensional surgical implant. The three-dimensional surgical implant includes a grip-type knit mesh defining pores and including a plurality of spiked naps extending from a surface thereof. The grip-type knit mesh is folded into a predetermined three-dimensional structure such that at least a portion of the spiked naps grip at least a portion of the pores to hold the three-dimensional structure of the surgical implant.

The present disclosure also is directed to a method of forming a three-dimensional surgical implant. The method includes: providing a grip-type knit mesh defining pores and including a plurality of spiked naps extending from a surface thereof; folding the grip-type knit mesh into a three-dimensional structure such that at least a portion of the pores and at least a portion of the spiked naps engage to fasten the surgical implant in the three-dimensional structure.

The present disclosure is also directed to a method of hernia repair. The method includes: providing a grip-type knit mesh defining pores and including a plurality of spiked naps extending from a surface thereof; folding the grip-type knit mesh into a three-dimensional structure such that at least a portion of the pores and at least a portion of the spiked naps engage to fasten the surgical implant into the three-dimensional structure; transferring said grip-type knit mesh into a body cavity having a hernia; and placing the grip-type knit mesh in the hernia to repair the hernia.

The present disclosure includes three-dimensional surgical implant. The three-dimensional surgical implant includes a grip-type knit mesh defining pores and including a plurality of spiked naps extending from a surface thereof. The three-dimensional surgical implant is formed by folding the grip-type knit mesh into a predetermined three-dimensional structure such that at least a portion of the spiked naps grip at least a portion of the pores to hold the three-dimensional structure of the surgical implant.

### BRIEF DESCRIPTION OF THE DRAWINGS

The foregoing objects and advantages of the disclosure will become more apparent from the reading of the following description in connection with the accompanying drawings, in which:

FIG. 1 is a top view of a grip-type knit mesh prior to forming a three-dimensional structure;

FIGS. 2A and B are perspective views of the grip-type knit mesh formed into three-dimensional structures; and

FIG. 3A-D are side cross-sectional views showing the use of the grip-type knit mesh in a hernia repair.

### DETAILED DESCRIPTION

The present disclosure relates to a grip-type knit mesh folded into a three-dimensional configuration. The grip-type knit mesh may be formed from biodegradable materials, non-biodegradable materials, or a combination of these. A grip-type knit mesh formed from a combination of biodegradable and non-biodegradable materials produces a semi-absorbable mesh resulting in less implanted mass while still providing a strong rigid support to maintain the long term integrity of the repair. A three-dimensional design formed with the grip portion facing outwards provides an additional means of fixation to secure the mesh to the tissue. The grip-type knit of the mesh also allows for formation of a specific shape to fit the patient's defect and the three-dimensional structure will be maintained without the need for stitching, gluing or pre-forming the mesh to a specific structure.

The present disclosure relates to devices, systems, and methods for minimally invasive surgeries such as, endoscopic, laparoscopic, arthroscopic, endoluminal and/or transluminal placement of a surgical patch at a surgical site. As used herein the term “surgical mesh” is used to refer to any three-dimensional grip-type implant for use in surgical procedures, such as, for example, meshes that do not require suturing to the abdominal wall. Although described herein with reference to a hernia mesh, the method of the disclosure may be used in any surgical repair. As used herein the term “laparoscopic deployment device” is used to refer to a deployment device that may be used during minimally invasive surgeries described above. Although described herein with reference to a minimally invasive surgery, the surgical mesh may also be used in open surgery.

Materials

As stated above, the fibers forming the grip-type knit mesh may be made from any fiber-forming biocompatible polymer. The biocompatible polymer may be synthetic or natural. The biocompatible polymer may be biodegradable, non-biodegradable or a combination of biodegradable and non-biodegradable. The term “biodegradable” as used herein is defined to include both bioabsorbable and bioresorbable materials. By biodegradable, it is meant that the materials decompose, or lose structural integrity under body conditions (e.g., enzymatic degradation or hydrolysis) or are broken down (physically or chemically) under physiologic conditions in the body such that the degradation products are excretable or absorbable by the body.

Representative natural biodegradable polymers which may be used include: polysaccharides, such as alginate, dextran, chitin, hyaluronic acid, cellulose, collagen, gelatin, fucans, glycosaminoglycans, and chemical derivatives thereof (substitutions and/or additions of chemical groups, for example, alkyl, alkylene, hydroxylations, oxidations, and other modifications routinely made by those skilled in the art); and proteins, such as albumin, casein, zein, silk, and copolymers and blends thereof, alone or in combination with synthetic polymers.

Synthetically modified natural polymers which may be used include: cellulose derivatives, such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt. These are collectively referred to herein as “celluloses.”

Representative synthetic degradable polymers suitable for use include: polyhydroxy acids prepared from lactone monomers, such as glycolide, lactide, caprolactone, ε-caprolactone, valerolactone, and δ-valerolactone, as well as pluronics, carbonates (e.g., trimethylene carbonate, tetramethylene carbonate, and the like); dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s; polyalkylene oxalates; polyoxaesters; polyanhydrides; polyortho esters; and copolymers, block copolymers, homopolymers, blends, and combinations thereof.

Some non-limiting examples of suitable non-bioabsorbable materials from which the fibers of the grip-type knit mesh may be made include: polyolefins, such as polyethylene and polypropylene including atactic, isotactic, syndiotactic, and blends thereof; polyethylene glycols; polyethylene oxides; ultra high molecular weight polyethylene; copolymers of polyethylene and polypropylene; polyisobutylene and ethylene-alpha olefin copolymers; fluorinated polyolefins, such as fluoroethylenes, fluoropropylenes, fluoroPEGSs, and polytetrafluoroethylene; polyamides, such as nylon and polycaprolactam; polyamines; polyimines; polyesters, such as polyethylene terephthalate and polybutylene terephthalate; aliphatic polyesters; polyethers; polyether-esters, such as polybutester; polytetramethylene ether glycol; 1,4-butanediol; polyurethanes; acrylic polymers and copolymers; modacrylics; vinyl halide polymers and copolymers, such as polyvinyl chloride; polyvinyl alcohols; polyvinyl ethers, such as polyvinyl methyl ether; polyvinylidene halides, such as polyvinylidene fluoride and polyvinylidene chloride; polyacrylonitrile; polyaryletherketones; polyvinyl ketones; polyvinyl aromatics, such as polystyrene; polyvinyl esters, such as polyvinyl acetate; copolymers of vinyl monomers with each other and olefins, such as etheylene-methyl methacrylate copolymers, acrylonitrile-styrene copolymers, ABS resins, and ethylene-vinyl acetate copolymers; alkyd resins; polycarbonates; polyoxymethylenes; polyphosphazine; polyimides; epoxy resins; aramids, rayon; rayon-triacetate; spandex; silicones; and combinations thereof.

Rapidly biodegradable polymers, such as poly(lactide-co-glycolide)s, polyanhydrides, and polyorthoesters, which have carboxylic groups exposed on the external surface as the smooth surface of the polymer erodes, may also be used. It should of course be understood that any combination of natural, synthetic, biodegradable and non-biodegradable materials may be used to form the grip-type knit mesh.

In embodiments, the naps of the grip-type knit mesh are formed from polylactic acid (PLA) and the mesh is formed from a monofilament polyester of polyethylene terephthalate (PET).

Bioactive Agents

The grip-type knit mesh may include a bioactive agent. The term “bioactive agent” as used herein, is used in its broadest sense and includes any substance or mixture of substances that have clinical use. Consequently, bioactive agents may or may not have pharmacological activity per se, e.g., a dye.

Alternatively, a bioactive agent could be any agent that provides a therapeutic or prophylactic effect, a compound that affects or participates in tissue growth, cell growth, cell differentiation, an anti-adhesive compound, a compound that may be able to invoke a biological action such as an immune response, or could play any other role in one or more biological processes. It is envisioned that the bioactive agent may be applied to the implant in any suitable form of matter, e.g., films, powders, liquids, gels and the like.

The bioactive agent may be bound to the grip-type knit mesh covalently, non-covalently, i.e., electrostatically, through a thiol-mediated or peptide-mediated bond, or using biotin-avidin chemistries and the like.

Examples of classes of bioactive agents, which may be utilized in accordance with the present disclosure include, for example, anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, platelet activating drugs, clotting factors and enzymes. It is also intended that combinations of bioactive agents may be used.

Anti-adhesive agents can be used to prevent adhesions from forming between the grip-type knit mesh and the surrounding tissues opposite the target tissue. In addition, anti-adhesive agents may be used to prevent adhesions from forming between the coated implantable medical device and the packaging material. Some examples of these agents include, but are not limited to hydrophilic polymers such as poly(vinyl pyrrolidone), carboxymethyl cellulose, hyaluronic acid, polyethylene oxide, poly vinyl alcohols, and combinations thereof.

Suitable antimicrobial agents which may be included as a bioactive agent include, for example, triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B may be included as a bioactive agent.

Other bioactive agents, which may be included include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents, such as coronary vasodilators and nitroglycerin; alkaloids; analgesics; narcotics such as codeine, dihydrocodeinone, meperidine, morphine and the like; non-narcotics, such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents, such as hormonal agents, hydrocortisone, prednisolone, prednisone, non-hormonal agents, allopurinol, indomethacin, phenylbutazone and the like; prostaglandins and cytotoxic drugs; chemotherapeutics, estrogens; antibacterials; antibiotics; anti-fungals; anti-virals; anticoagulants; anticonvulsants; antidepressants; antihistamines; and immunological agents.

Other examples of suitable bioactive agents which may be included in the grip-type knit mesh include, for example, viruses and cells, including stem cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, α-IFN and γ-IFN); erythropoietin; nucleases; tumor necrosis factor; colony stimulating factors (e.g., GCSF, GM-CSF, MCSF); insulin; anti-tumor agents and tumor suppressors; blood proteins such as fibrin, thrombin, fibrinogen, synthetic thrombin, synthetic fibrin, synthetic fibrinogen; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); bone morphogenic proteins; TGF-B; protein inhibitors; protein antagonists; protein agonists; nucleic acids, such as antisense molecules, DNA, RNA, RNAi; oligonucleotides; polynucleotides; and ribozymes.

Mesh Structure

The knit forming the mesh may include a monofilament sheet forming, on at least a portion of at least one face of the knit, spiked naps which protrude with respect to the sheet. In embodiments, the naps each have a substantially rectilinear body and, at the free end of this body, a head of greater width than that of the body.

This knit can be formed using a thermofusible monofilament to form a monofilament sheet, forming outer loop-shaped meshes in the sheet, and then partially fusing the monofilament.

The length of the spiked naps is defined so as to penetrate and fasten to the porous textile structure of the knit in a limited manner, that is to say without emerging from the other face, for example when the nap portion of a knit including spiked naps is applied against a porous portion, of the same knit or of a different knit.

In embodiments, the monofilament forming the spiked naps can have a diameter from about 0.05 mm to about 0.15 mm, in embodiments a diameter of over 0.10 mm. Each spiked nap can have a length of from about 1 mm and about 2 mm, in embodiments a length of about 1.5 mm. The density of the spiked naps can be from about 50 and about 90 naps per square centimeter, in embodiments from about 65 and about 75 naps per square centimeter. Suitable grip-type knit meshes and methods for making them are disclosed in U.S. Pat. No. 7,331,199, the disclosure of which is incorporated by reference herein in its entirety.

The textile structure of the knit may include two faces, one with the spiked naps, and one with open pores, which for example may have a diameter of from about 1 mm and about 3 mm. For example, this structure can include several sheets of interlaced yarns, which together form a layered structure. When interlaced yarns are used, the layered structure may be composed, for example, of three sheets: an intermediate sheet of yarn distributed to form a zigzag openwork pattern between the columns of meshes; a front sheet of yarn distributed to form a chain stitch; and a rear sheet of monofilament placed in partial weft under the chain stitch and “thrown onto” the needle not forming a chain stitch, this sheet may include the spiked naps.

When a grip-type knit is applied, with spiked naps to the front, onto a surface of a porous prosthetic knit during manipulation into a three-dimensional configuration, the spiked naps engage into the mesh and between the multifilament yarns of the porous knit and fasten the grip-type knit onto the porous knit. This fastening, effective even in a liquid environment, is sufficient to secure the mesh in the desired three-dimensional configuration, and to offer mechanical resistance to tangential stresses, while at the same time permitting unfastening of the grip-type knit in order to adjust its position in relation to the element lying underneath, if desired.

In embodiments, the porous knit portion of the mesh may include size markings. The size markings may indicate the location into which the grip-type knit may be secured to the porous knit during manipulation into a three-dimensional configuration in order to obtain three-dimensional structures (e.g., cones) of various sizes. The markings may be any type of marking as is known in the art. For example, a dye or colorant may be placed (e.g., printed) at specific locations on the porous knit. As another example, a colored yarn may be woven into specific locations of the porous knit. Those skilled in the art will readily envision other ways of applying suitable markings to the mesh.

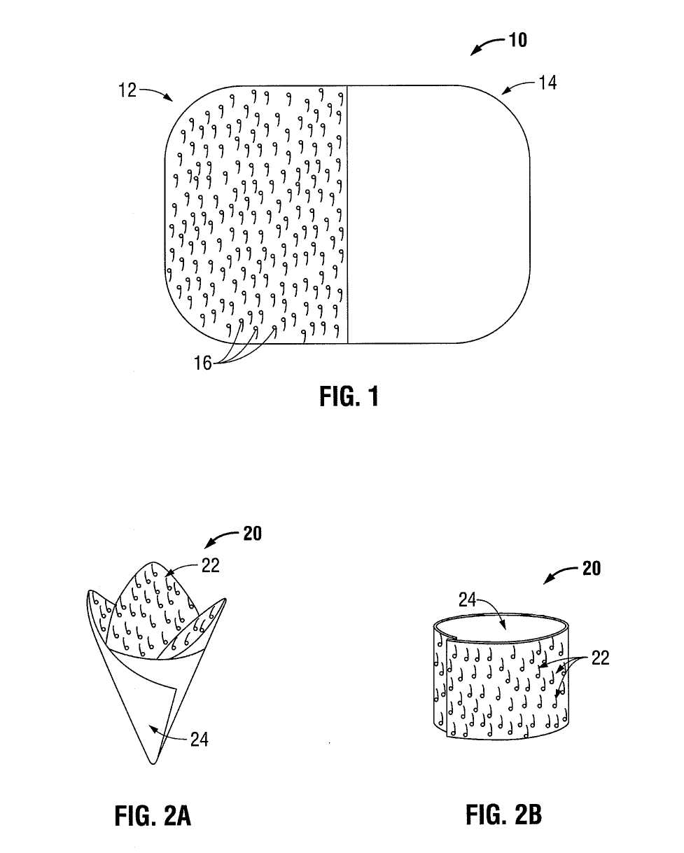

Referring now in specific detail to the drawings, in which like numbers identify similar or identical elements, FIG. 1 is an illustration of a grip-type knit mesh prior to forming a three-dimensional structure. The grip-type knit mesh 10 includes sides 12 and 14. Side 12 includes naps 16 which grip into the open pore structure of side 14. Although sides 12 and 14 are each shown as covering half of the mesh, the naps 16 may cover less or more of the mesh. It is also envisioned that the naps can cover an entire side of a mesh.

Three-Dimensional Structure

As stated above, the spiked naps grip onto the porous portion of the mesh in such a manner as to be secure yet capable of being detached and reattached as necessary. The grip-type knit mesh may be formed or folded into a three-dimensional structure. For example, the knit may be formed or folded into a cone, cylinder, triangle, square, and the like. In embodiments, the three-dimensional structure can be held together by using the naps engaged with the open pore structure wherever there is overlap.

The naps of the grip-type knit mesh may face inward or outward in relation to the three-dimensional structure of the mesh. When the naps face outward, they provide a means of affixing the mesh to the surrounding tissue. In embodiments, the three-dimensional structure can be formed from the grip-type knit during production, i.e., without the use of the naps to hold the structure into a shape.

In embodiments, the surgeon can form the grip-type knit mesh into the desired shape prior to using the mesh in situ. In embodiments, markings on the grip-type knit mesh can provide guidance as to how to fold or form the mesh into a three-dimensional structure.

FIGS. 2A and B show different configurations of the three-dimensional mesh of the present disclosure. Mesh 20, when planar, has a nap portion 22 on one side and an open pore portion 24 on the other side. When folded into a conical structure (FIG. 2A), the nap portion 22 may face inward or outward. Mesh 20 may also be folded into a conical formation (FIG. 2B) with nap portion 22 facing outward from open pore portion 24.

### Methods of Use

In accordance with the present disclosure the three-dimensional grip-type knit mesh may be used in either minimally invasive or open surgery. A minimally invasive method of treating a hernia includes: making an incision in the abdominal wall close to the herniated area; making a subcutaneous cut, through the incision, over and surrounding the area of the hernia; inserting a three-dimensional grip-type knit mesh through the incision using a laparoscopic device; and inserting the three-dimensional grip-type knit mesh into the hernia

Thus, a mesh according to the present disclosure can be inserted through a small incision (e.g., from about 1 cm to about 2 cm in length) in the abdominal cavity. In embodiments, a hernia region is reached using an anterior surgical approach. The grip-type surgical mesh is formed into a three-dimensional structure by fastening the grip portion to the porous portion of the mesh. The three-dimensional structure may mirror the three-dimensional structure of the defect. The mesh is then inserted through the opening in the tissue wall until the base lies flush with or slightly beyond the defect. When the grip portions are facing outward they will grip to the tissue securing the mesh within the tissue. The mesh thus conforms to the shape of the defect and adheres to the surrounding tissue in such a way as to secure the mesh to the tissue. It is also contemplated that a surgical fastener is used to attach the mesh to the surrounding tissue. In embodiments where the naps of the grip-type knit mesh are formed from a biodegradable material such as, for example, a polylactic acid (PLA) and the mesh is formed from a non-biodegradable material such as, for example, monofilament polyester of polyethylene terephthalate (PET), the naps of the mesh will degrade over time while the non-degradable portion of the mesh remains to provide stability to the mesh. This results in less foreign material left in the patient.

A separate flat grip-type knit mesh may also be adhered to the surrounding tissue.

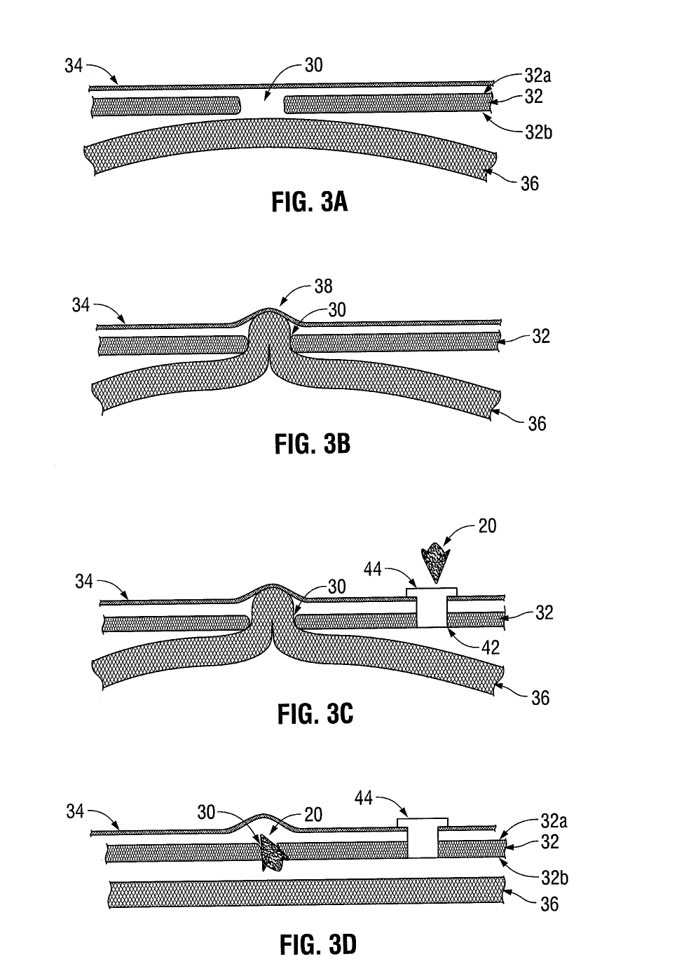

Referring now to FIGS. 3A-3D, a method of using a three-dimensional grip-type knit mesh to perform a surgical repair procedure is shown and described. With reference to FIG. 3A, a hernia may involve a tear 30, in the abdominal wall 32. Abdominal wall 32 is defined by an external side 32a and peritoneum 32b. A surface tissue 34, which covers the external side 32a of abdominal wall 32, may or may not be immediately affected by this tear 30. An internal organ 36 located below the peritoneum 32b of the abdominal wall 32 may not protrude until some form of exertion or use of the muscle located at the abdominal wall 32 forces the internal organ 36 into the tear 30. Depending on the size and location of the tear 30, exertion may not be needed to cause the organ to protrude. As shown in FIG. 3B, a hernia occurs when internal organ 36 protrudes into the tear 30 of abdominal wall 32. Oftentimes the protrusion creates a bulge 38 in the surface tissue 34.

In order to correct the defect, as depicted in FIG. 3C, an incision 42 is made through the abdominal wall 32 in close proximity to tear 30 and a three-dimensional grip-type knit mesh 20 is inserted using a trocar 44 or similar laparoscopic device. As shown in FIG. 3D, a three-dimensional grip-type knit mesh 20 is then placed in the tear 30 from the peritoneum 32b of the abdominal wall 32. The naps 16 attach to the abdominal wall 32 and allow the mesh 20 to fill the tear 30 in the abdominal wall 32 and return the internal organ 36 to its original location.

While the above description contains many specifics, these specifics should not be construed as limitations on the scope of the present disclosure, but merely as exemplifications of preferred embodiments thereof. Those skilled in the art will envision many other possible variations that are within the scope and spirit of the present invention.

What is claimed is:

1. A three-dimensional surgical implant comprising:
a grip-type knit mesh defining pores and including a plurality of spiked naps extending from a surface thereof the grip-type knit mesh being folded into a predetermined three-dimensional structure such that at least a portion of the spiked naps grip at least a portion of the pores to hold the three-dimensional structure of the surgical implant.

2. The three-dimensional surgical implant of claim 1, wherein the grip-type knit mesh comprises materials selected from the group consisting of biodegradable, non-biodegradable, and combinations thereof.

3. The three-dimensional surgical implant of claim 1, wherein the material defining the pores is non-biodegradable.

4. The three-dimensional surgical implant of claim 3, wherein the material defining the spiked naps is biodegradable.

5. The three-dimensional surgical implant of claim 4, wherein the biodegradable material defining the spiked naps is selected from the group consisting of polylactic acid, polyglycolic acid, poly(lactide-co-(ε-caprolactone)), poly(glycolide-co-(ε-caprolactone)), poly(lactide-co-glycolide), and combinations thereof.

6. The three-dimensional surgical implant of claim 3, wherein the non-biodegradable material defining the pores is selected from the group consisting of polyethylenes, polypropylenes, ultra high molecular weight polyethylene, and combinations thereof.

7. The three-dimensional surgical implant of claim 1, wherein the predetermined three-dimensional structure is conical.

8. The three-dimensional surgical implant of claim 1, wherein the predetermined three-dimensional structure is cylindrical.

9. The three-dimensional surgical implant of claim 1, further comprising a bioactive agent.

10. The three-dimensional surgical implant of claim 9, wherein the bioactive agent is selected from the group consisting of anesthetics, analgesics, and antispasmodics.

11. The three-dimensional surgical implant of claim 1, wherein the spiked naps are each from about 1 mm to about 2 mm in length.

12. The three-dimensional surgical implant of claim 1, wherein the density of spiked naps on the grip-type knit mesh is from about 50 to about 90 spiked naps per square centimeter.

13. A method of forming a three-dimensional surgical implant comprising the steps of:
providing a grip-type knit mesh defining pores and including a plurality of spiked naps extending from a surface thereof;
folding the grip-type knit mesh into a three-dimensional structure such that at least a portion of the pores and at least a portion of the spiked naps engage to fasten the surgical implant in the three-dimensional structure.

14. The method of claim 13, further comprising the step of unfastening at least a portion of the spiked naps from at least a portion of the pores.

15. The method of claim 14, further comprising the step of adjusting the three-dimensional structure of the grip-type knit mesh.

16. The method of claim 15, further comprising the step of refastening at least a portion of the spiked naps to at least a portion of the pores.

17. The method of claim 13, wherein the step of folding the grip-type knit mesh into the three-dimensional structure forms a three-dimensional structure having an outward face and an inward face and wherein the spiked naps are located on the outward face of the three-dimensional structure.

18. The method of claim 13, wherein the step of folding the grip-type knit mesh into the three-dimensional structure forms a three-dimensional structure having an outward face and an inward face and wherein the spiked naps are located on the inward face of the three-dimensional structure.

19. A method of hernia repair comprising:
providing a grip-type knit mesh defining pores and including a plurality of spiked naps extending from a surface thereof;
folding the grip-type knit mesh into a three-dimensional structure such that at least a portion of the pores and at least a portion of the spiked naps engage to fasten the surgical implant into the three-dimensional structure;
transferring said grip-type knit mesh into a body cavity having a hernia; and
placing the grip-type knit mesh in the hernia to repair the hernia.

20. The method of claim 19, wherein the step of folding comprises folding the grip-type knit mesh into a cone.

21. The method of claim 19, wherein the step of folding comprises folding the grip-type knit mesh into a cylinder.

22. The method of claim 19, wherein the step of folding the grip-type knit mesh into a three-dimensional structure forms a three-dimensional structure having an outward face and an inward face wherein the spiked naps are located on the outward face of the three-dimensional structure such that at least a portion of the spiked naps are positioned to engage tissue thereby fastening the grip-type knit mesh to surrounding tissue.

23. The method of claim 19, wherein the step of folding the grip-type knit mesh into a three-dimensional structure forms a three-dimensional structure having an outward face and an inward face wherein the spiked naps are located on the inward face of the three-dimensional structure.

24. The method of claim 19, wherein the step of folding the grip-type knit mesh into a three-dimensional structure occurs within the body cavity.

25. The method of claim 23, wherein the step of fastening the grip-type knit mesh comprises fastening the grip-type knit mesh to surrounding tissue with a surgical fastener.

26. The method of claim 19, wherein the step of transferring said grip-type knit mesh into a body cavity is accomplished laparoscopically.

27. A three-dimensional surgical implant comprising:
a grip-type knit mesh defining pores and including a plurality of spiked naps extending from a surface thereof whereby the three-dimensional surgical implant is formed by folding the grip-type knit mesh into a predetermined three-dimensional structure such that at least a portion of the spiked naps grip at least a portion of the pores to hold the three-dimensional structure of the surgical implant.
